(19) United States
(12) Patent Application Publication
(10) Pub. No.: US 2004/0241741 A1
(43) Pub. Date: Dec. 2, 2004

(54) Identification of proteins using a physical parameter and accurate amino acid content

(76) Inventors: Rodney Levine, Rockville, MD (US); Mary Ann Robinson, Rockville, MD (US)

Correspondence Address: Birch, Stewart, Kolasch & Birch, LLP, 8110 Gatehouse Rd, Suite 500 East, P.O. Box 747, Falls Church, VA, 22040-0747, US

(21) Appl. No.: 10/446,865

(22) Filed: May 29, 2003

(51) Int. Cl. G01N 33/53; G06F 19/00; G01N 33/48; G01N 33/50

(52) U.S. Cl. 435/7.1; 702/19

(57) ABSTRACT

An unidentified purified protein can be uniquely identified by combining one or more physical parameters of the protein and an accurate amino acid content. The protein can be identified by input of the one or more physical parameters and the amino acid content into an interface that outputs data from a protein database.

### BACKGROUND OF INVENTION

1. Field of the Invention

The instant invention relates to being able to uniquely identify an isolated protein by using the physical properties of the protein and submitting those physical properties as a query into a protein database, thereby obtaining a uniquely identified protein.

2. Description of Related Art

Shaw (Shaw, G., Proc. Natl. Acad. Sci. Vol. 90, pp. 5138-5142, (1993)) discloses a method of identifying a list of possible proteins when a protein that has been isolated is unknown. Shaw's method uses the relative amino acid content in the protein to identify a group of proteins, which should include the actual identity of the protein. The method employed by Shaw to determine relative amino acid content includes digesting the protein, reacting the protein with ninhydrin, and using ultraviolet-visible spectroscopy to measure the relative amounts of ninhydrin attached to 18 of the naturally occurring amino acids. This method gives the relative amounts of each of these 18 naturally occurring amino acids but fails to determine the amounts of tryptophan and cysteine. The data obtained from the relative amounts of these 18 amino acids combined with one or more other physical properties of the protein is then input into a protein data base to get a list of possible proteins that the previously unidentified protein might be. The method of Shaw utilizes a scoring system to narrow the myriad possibilities down to a list for what is most likely to be the identity of the protein. The method of Shaw, although useful, is unable to uniquely identify a single protein. Moreover, as the sizes of protein databases increase with the relatively recent revolution in mapping the genetic code, the method employed by Shaw is less likely to identify an unique protein. Shaw's method also suffers from the drawback of data inaccuracy due to the limitations in the ninhydrin method, which precludes the unique identification of the protein. Further, the method of Shaw suffers the drawback of having to digest the protein in order to determine the relative amounts of amino acids.

Wilkins et al. (Wilkins M. R., Pasquali C., Appel R. D., Ou K., Golaz O., Sanchez J. C., Yan J. X., Gooley A. A., Hughes G., Humphery-Smith I., Williams K. L., Hochstrasser D. F., Biotechnology Vol. 14(1), pp. 61-65 (January 1996)) disclose the separation and identification of proteins by two-dimensional (2-D) electrophoresis. The method of Wilkins et al. can be used for protein-based gene expression analysis. In this reference, single protein spots, from polyvinylidene difluoride blots of micropreparative E. coli 2-D gels, were identified by matching their amino acid composition, estimated pI and molecular weight against all E. coli entries in the SWISS-PROT database. Thirty proteins from an E. coli 2-D map were analyzed and their identities assigned. Three of the proteins were unknown. By protein sequencing analysis, 20 of the 27 proteins were correctly identified. Importantly, correct identifications showed unambiguous “correct” score patterns. However, incorrect protein identifications also showed distinctive score patterns; these proteins must be identified by other means. Wilkins' techniques allow large-scale screening of the protein complement of simple organisms, or tissues in normal and disease states.

Levine et al. (Levine R. I., and Federici M. M., Biochemistry, Vol. 21, pp. 2600-2606, (1982)) disclose a method of accurately identifying the amounts of the aromatic amino acids tryptophan, tyrosine and phenylalanine in a protein. The method involves using second derivative ultraviolet-visible spectroscopy (d²absorbance/d²(nm)) in the ultraviolet region of the electromagnetic spectrum. Employing second derivative spectroscopy essentially eliminates the contribution from light scattering and disulfide bond absorbance and allows one to accurately quantify the amounts of tyrosine, tryptophan and phenylalanine. Molar absorbtivities (εₘ) of 110.6 at 248 nm for phenylalanine, 1330 at 276 nm for tyrosine, and 5620 at 281 nm for tryptophan were calculated, which could be used to accurately determine the amounts of phenylalanine, tyrosine, and tryptophan in proteins. The Levine et al. reference is herein incorporated by reference in its entirety.

It is known in the art that the amount of cysteine and its homologs in a protein can be determined by spectrofluorometric analysis. Liang et al. (Liang S. C., Wang H., Zhang Z. M., Zhang X., Zhang H. S., Spectrochim Acta A Mol Biomol Spectrosc. Vol. 58(12), pp. 2605-11 (October 2002)) disclose the spectrofluorometric determination of cysteine by 5-maleimidyl-2-(m-methylphenyl)benzoxazole. Tcherkas et al. (Tcherkas Y. V., Denisenko A. D., J Chromatogr A., Vol. 913(1-2), pp. 309-13 (April 2001), disclose the simultaneous determination of several amino acids, including homocysteine, cysteine and glutamic acid, in human plasma by isocratic reversed-phase high-performance liquid chromatography with fluorimetric detection; Liang and Tcherkas et al. are herein incorporated in their entirety by reference.

It is desired to be able to uniquely identify a purified protein by a method that is cheap, fast and accurate. The instant invention achieves this goal.

### BRIEF DESCRIPTION OF THE SEVERAL VIEWS OF THE DRAWINGS

FIG. 1 is an elution profile of an HPLC of four proteins from cow. The elution profile shows the elution of albumin, carbonic anhydrase II, methionine enkephalin, and ribonuclease, in order of their elution.

FIG. 2 displays a sample page of a web interface used to input data for a look-up in the protein databank.

FIG. 3 displays an example of an input page that allows the user to input physical parameters to initiate the search to identify the protein.

FIG. 4 displays an example of an output page that identifies the protein.

FIG. 5 displays the distributions of molecular weights (MW) of proteins in the Swiss-Prot database. The molecular weight is along the ordinate and the relative percentage of the proteins with a given molecular weight is along the abscissa.

FIG. 6 displays the distributions of the tyrosine to tryptophan ratio of proteins in the Swiss-Prot database. The tyrosine to tryptophan ratio is along the ordinate and the relative percentage of the proteins with a given tyrosine to tryptophan ratio is along the abscissa.

FIG. 7 displays the distributions of the tyrosine to phenylalanine ratio of proteins in the Swiss-Prot database. The tyrosine to phenylalanine ratio is along the ordinate and the relative percentage of the proteins with a given tyrosine to phenylalanine ratio is along the abscissa.

FIG. 8 displays the distributions of the isoelectric points of proteins in the Swiss-Prot database. The isoelectric point of each respective protein is along the ordinate and the relative percentage of the proteins with a given isoelectric point is along the abscissa.

### DETAILED DESCRIPTION OF THE INVENTION

A purified protein can be uniquely identified by combining a physical parameter of the protein and an accurate amino acid content by submitting a query containing the physical parameter and the amino acid content into a protein data base.

In particular, the inventors of the instant invention have found that by combining an accurate determination of the amount of at least one amino acid per molecule and a physical parameter of that protein, an accurate and unique determination of that protein can be made by submitting a query containing that data into a protein data base. The database will output a correct and unique protein identity.

Thus, the instant invention includes as one embodiment a method for identifying a purified protein comprising determining an amount per molecule of at least one amino acid type but fewer than 18 amino acid types, determining a physical parameter of the protein, submitting a query to a protein database including the at least one physical parameter of the protein and the amount per molecule of at least one amino acid type, and identifying the protein based on that physical parameter of the protein and the amount per molecule of at least one amino acid type, wherein the determination of the physical parameter of the protein and the determination of the amount of at least one amino acid are performed in any order.

In particular, the instant invention includes as one embodiment a method for identifying a purified protein comprising determining an amount per molecule of at least one amino acid type but fewer than 18 amino acid types, determining a mass of the protein, submitting a query to a protein database including the mass of the protein and the amount per molecule of at least one amino acid type, and identifying the protein based on the mass of the protein and the amount per molecule of at least one amino acid type, wherein the determination of the mass of the protein and the determination of the amount of at least one amino acid are performed in any order.

Another embodiment of the instant invention includes a method for identifying a purified protein comprising determining an amount per molecule of at least one amino acid type but fewer than 18 amino acid types, determining an isoelectric point of the protein, submitting a query to a protein database including the isoelectric point of the protein and the amount per molecule of at least one amino acid type, and identifying the protein based on the isoelectric point of the protein and the amount per molecule of at least one amino acid type, wherein the determination of the isoelectric point of the protein and the determination of the amount of at least one amino acid are performed in any order.

In a preferred embodiment of the above methods, the at least one amino acid type comprises at least one aromatic amino acid or cysteine, wherein the aromatic amino acid is selected from the group consisting of phenylalanine, tyrosine, histidine, tryptophan, and modifications of any of these amino acids.

The mass of the protein can be determined by any number of means including, but not limited to, mass spectroscopy, gel electrophoresis, size exclusion chromatography (gel filtration method under denaturing conditions), intrinsic viscosity, sedimentation-diffusion centrifugation, sedimentation equilibrium centrifugation, viscoelasticity, thin layer chromatography, NMR spectroscopy (chemical shift anisotropy), air turbine centrifugation, and other spectroscopic methods. Preferred methods include a gel filtration method under denaturing conditions, gel electrophoresis, and mass spectroscopy with matrix assisted laser desorption ionization (MALDI), continuous or pulsed electrospray ionization, ionspray, thermospray, or massive cluster impact mass spectrometry. The detection format for mass spectroscopy includes linear time-of-flight (TOF), reflectron time-of-flight, single quadruple, multiple quadruple, single magnetic sector, multiple magnetic sector, Fourier transform ion cyclotron resonance, ion trap, and combinations thereof (such as MALDI-TOF spectrometry). MALDI-TOF and electrospray mass spectroscopy are particularly preferred.

The isoelectric point can be determined by any of a number of methods including, but not limited to, isoelectric focusing, calculating the pI by the amino acid composition, and any other method that is recognized by one of skill in the art.

It should be recognized that the method of the instant invention allows for variation in the mass of the protein. In other words, the purified protein can still be accurately, effectively, and uniquely identified even with variations in the mass of the purified protein. Thus, it should be understood that even if acetylation, myristolyation, or phosphorylation of the protein takes place, or any other covalent modification takes place, the purified protein can still be accurately and uniquely identified. Tables 2 and 3 show the robustness of the invention to uncertainties in the parameters used in the instant method.

The amount per molecule of at least one amino acid type is most preferably determined by second derivative UV-visible spectroscopy or by spectrofluorometric methods. These accurate methods are described in the Examples. The accuracy of these methods are such that when determining an amount per molecule of at least one amino acid type, the deviation from the actual amino acid amount per molecule of at least one amino acid type is 15% or less, with a deviation that is preferably 10% or less, more preferably 8% or less, even more preferably 6% or less, and most preferably 5% or less.

Protein databases that can be used include the Brookhaven database, Swiss-Prot database, GeneBank database, Prosite database, and the Research Collaboratory for Structural Bioinformatics database. Alternatively, one might construct a database/databank for use employing the method of the instant invention. The Swiss-Prot database is preferred.

The instant invention preferably utilizes full-length protein. By full-length protein, it is meant protein as purified in which no peptide bonds, and in some embodiments, no covalent bonds, have been cleaved after purification.

In another embodiment of the instant invention, a ratio in the amounts of two or more amino acids can be used as a parameter to input into the protein database to aid in uniquely identifying the purified protein. Preferred embodiments include the ratios of the following amino acids. C/F, F/C, F/W, W/F, F/Y, Y/F, F/H, H/F, C/W, W/C, C/Y, Y/C, C/H, H/C, W/Y, Y/W, W/H, H/W, Y/H, and H/Y. However, it should be recognized that any ratio of amino acids can be used.

It will be recognized by one of ordinary skill in the art that should a unique protein not be identified, the submission of a query with additional physical parameters can be made, which will aid in uniquely identifying the protein. Thus, in particular, it should be recognized that the combination of the amount(s) of at least one amino acid per molecule and of any other physical parameter can be used to uniquely identify the protein. Additional physical parameters can be added should the combination of the amount(s) of at least one amino acid per molecule and of one other physical parameter not be effective to uniquely identify the protein. For example, any combination of the amount(s) of at least one amino acid per molecule, and one or more of the other physical parameters such as mass of the protein, intrinsic viscosity, or isoelectric point can be utilized.

The instant invention is also advantageous in that very small amounts of protein can be used to uniquely identify that protein. The inventors conservatively estimate that 1 picomole of protein is sufficient to uniquely identify the protein. This amount is calculated as follows. First, one should use the smallest commercially available cell for the spectrophotometer, that is an HPLC diode array detector with a 0.50 microliter volume (model 1100) sold by Agilent (Palo Alto, Calif.). The inventors know that an absorbance of at least 0.100 absorbance units is sufficient because Levine et al. (Levine R. I., and Federici M. M., Biochemistry, Vol. 21, pp. 2600-2606, (1982)) showed this amount gives confident results. Assuming that the molar absorbtivity of a protein is 50,000 (a common assumption, as in Methods in Enzymology, Vol. 233, p. 346 (1994)), a solution with absorbance 0.10 has a protein concentration of 2 micromolar. Thus, the 0.5 microliter cell contains 1 pmol.

One can easily improve the sensitivity. For example, a manufacturer could make the cell 10 cm in length instead of the standard 1 cm, giving a ten-fold increase in sensitivity. Another approach is simply to use signal averaging, that is, acquiring more spectra and average them, thus improving the signal/noise ratio. The improvement in signal is directly proportional to the square of the number of spectral scans. Finally, microfluidics could reduce the volume of sample required by one to two orders of magnitude. Microfluidic techniques are known in the art and the equipment can be obtained from Gyros US, Inc. (Monmouth Junction, N.J.). Thus, achieving an overall 100-fold improvement is quite reasonable, bringing the conservative estimate down to 10 femtomoles (i.e., 10×10⁻¹⁵ moles) of protein as a feasible sample size.

This amount is less than the amount necessary to identify a protein by MALDI-MAS. Yale University offers the possibility of identifying proteins by MALDI-MAS wherein the proteins are usually submitted as Coomassie Blue stained gel bands/spots from 1D/2D SDS polyacrylamide gels following MALDI-MS of a (usually) small aliquot of the resulting in gel tryptic digest. The limit of sensitivity is in the 10-50 fmol range (assuming most of the digest is committed to MALDI-MS) and the resulting monoisotopic peptide masses are then submitted to one or more peptide mass search algorithms to identify protein(s) that are present. (W. M. Keck Facility, Yale University, 301 BCMM, P.O. Box 9812, New Haven, Conn.). However, the cost of the mass spectrometer is on the order of half a million dollars, substantially more than the costs involved in the instant invention. Moreover, the method offered by Yale University suffers the drawback of having to use tryptic digests, which is not the case in the instant invention.

The instant invention is exemplified by the following examples, which are not meant to limit the scope of the invention but are to be used as illustrative examples.

### EXAMPLES

### Example 1

Protein Identification

One application of the invention is the identification of proteins in a mixture, which are separated by reverse phase HPLC. To assess the method's usefulness, the inventors made a mixture of four bovine proteins, purchased from the Sigma Chemical Company (St. Louis, Mo.):

1) Albumin

2) Carbonic anhydrase II

3) Methionine enkephalin

4) Ribonuclease

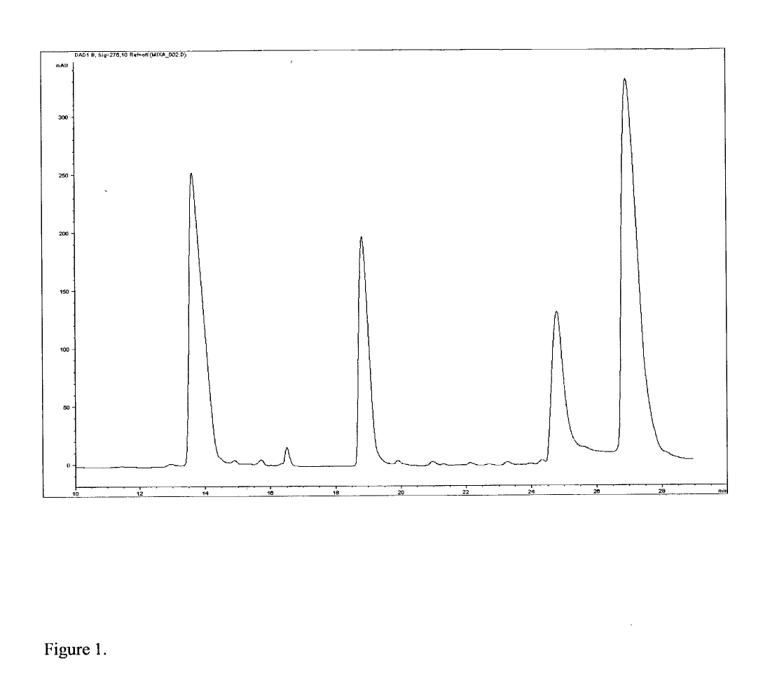

Equal volumes of each protein (1 mg/ml dissolved in phosphate buffered saline) were mixed together. A sample was applied to a reverse phase column (Vydac 218TP5205 from Grace Vydac (Columbia, Md.)). The proteins were eluted by a linear gradient of acetonitrile. Four peaks were observed in the chromatogram, and these were collected in separate tubes of 100 microliters. See FIG. 1.

Half of a microliter of the solution was used to estimate molecular weight by MALDI-TOF mass spectrometry. The remainder of the fraction was dried, re-dissolved in 20 microliters of 6M guanidine solution, and used for amino acid determination by a spectral deconvolution procedure. Spectra were taken with a Hewlett Packard Model 8450A spectrophotometer. This spectrophotometer permits parallel (simultaneous) acquisition of absorbances at 401 specific wavelengths. The entire spectrum can be measured twice, the results averaged, and the spectrum displayed on the cathode ray screen in about 1 s. Typically, 20 individual spectra are taken over a 10 s period. The spectrophotometer also includes a microprocessor that allows rapid calculation of derivatives of the absorbance spectra. The microprocessor also implements a multicomponent analysis method that takes advantage of estimates of precision available with the averaged spectra. The instrument was balanced with 6 M guanidine solution to give an automatic baseline correction. Then each of the second derivative spectra for tyrosine, phenylalanine and tryptophan were taken for the 4 proteins. For each of the 4 peaks the inventors obtained estimates of the molecular weight (by MALDI-TOF mass spectroscopy (see Table 1). The ratios of tyrosine to tryptophan and tyrosine to phenylalanine (from the Hewlett Packard Model 8450A spectrophotometer) were determined.

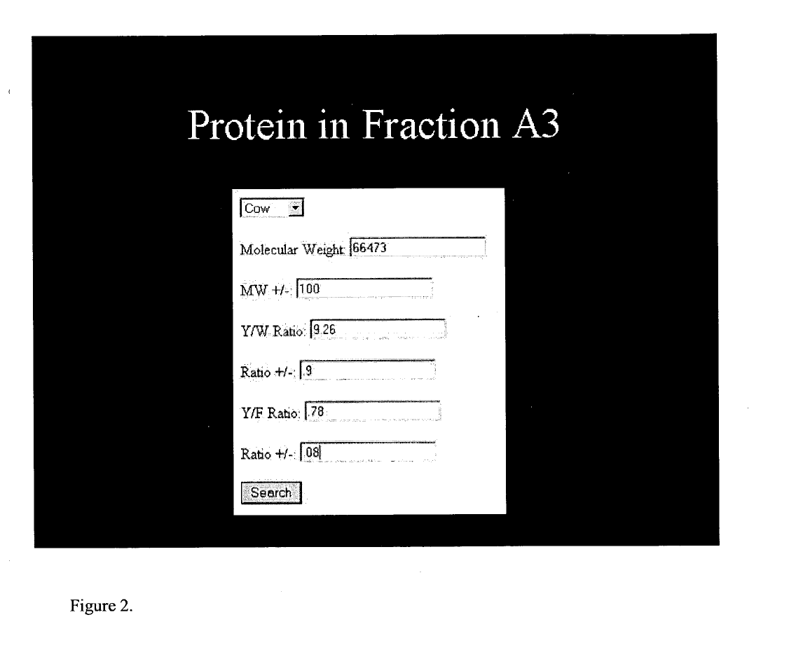

These values (ratios of tyrosine to tryptophan and tyrosine to phenylalanine and the absolute amounts of tyrosine, tryptophan and phenylalanine) were entered individually into the database searching program, which interrogated the SWISS-PROT database for possible matches. Table 1 shows the Y/W and Y/F ratios, the molecular weight and the correctly identified protein. Example 3 describes the design of the computer program and a screen snapshot is shown of the entry form for the third peak that eluted from the HPLC in FIG. 2.

For all 4 fractions, only a single protein from the database matched, and the match correctly identified each protein. See Table 1 for the identification of the protein.

| | Tyrosine/ | Tyrosine/ | Molecular | |
|---|---|---|---|---|
| Fraction | Tryptophan | Phenylalanine | Weight | Identification |
| 1 | 23.84 | 0.86 | 573.5 | Methionine |
| | | | | enkephalin |
| 2 | 366.75 | 2.13 | 13,676 | Ribonuclease |
| 3 | 9.26 | 0.78 | 66,473 | Albumin |
| 4 | 1.09 | 0.66 | 28,998 | Carbonic |
| | | | | anhydrase II |

Table 1 shows the correct identification of all of the proteins from the protein mixture based on molecular weight and second derivative UV-Visible spectra.

### Example 2

Identification of a Purified Protein Using the Amount of Cysteine and Glutamic Acid

A protein is purified that has cysteine and glutamic acid in it. The amount of cysteine (and any of the homologs of cysteine) and glutamic acid are determined following the methods of Liang et al. (Liang S. C., Wang H., Zhang Z. M., Zhang X., Zhang H. S., Spectrochim Acta A Mol Biomol. Spectrosc. Vol. 58(12), pp. 2605-11 (October 2002)) and Tcherkas et al. (Tcherkas Y. V., Denisenko A. D., J Chromatogr A., Vol. 913(1-2), pp. 309-13 (April 2001), respectively. The ratio of cysteine to glutamic acid is calculated and this ratio is combined with a physical parameter, such as the isoelectric point or intrinsic viscosity and input into the Swiss-Prot database following the procedure as enumerated below in Example 3.

### Example 3

Design of Computer Program for Accessing Swiss-Prot Database.

Design and construction of maryquery: A database for protein identification.

A web-site interface was generated that allows the input of physical data related to a protein at the interface site. The interface uses information from a protein database to generate an output containing the uniquely identified protein. Please see http://www.ncbi.nlm.nih.gov/projects/core_bio/core_home/cgi/maryguery.html. One embodiment of the interface site is represented in FIG. 2. In this embodiment, the user inputs data into the interface site by use of a mouse and typing in the physical parameters of the known parameters as well as uncertainty values into the interface site. Using the mouse and clicking on search will output the identity of the protein. It will be recognized by those of skill in the art that this is only one embodiment of the invention. One could conceive of additional physical parameters that could be added to this interface page (including other parameters discussed in this application) that are all within the scope of the instant invention. Further, the order of the input or the appearance of the interface page can be modified, which are all within the scope of the instant invention.

In one embodiment, the Swiss-Prot Protein Knowledgebase was down loaded from the ftp site (at ftp://ftp.ebi.ac.uk/pub/databases/swissprot/) of Swiss-Prot. These are flat files of extensively annotated protein sequences. The inventors used the version swisspro40 and added the entries contained in the November 2002 update file. All species are included. It should be recognized that any other protein database can be used.

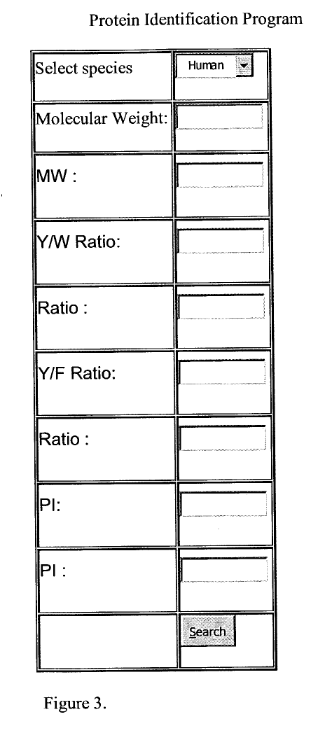

A perl script was written to extract information from the file, process it and produce look-up tables for human, mouse, E. coli, rat and cow. A web-based interface was created and linked to the files for each species. See FIG. 3 for a particular example. This program is operated by entering measured values for Molecular weight, Y/W, Y/F, (tyrosine/tryptophan and tyrosine/phenylalanine, respectively) and pI along with uncertainty factors to establish the range of data to be included in the search.

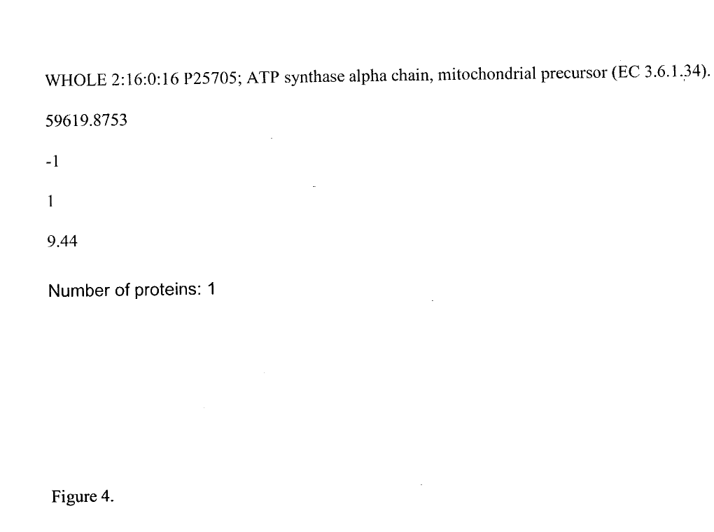

The search is initiated and the results are output on another page. See FIG. 3 for the page on which the search is initiated and see FIG. 4 for an example of the output page.

The following description describes the output page as exemplified in FIG. 4. The first line of the output page displays information about the entry. “Whole” or “Chain” refers to whether or not the leader sequence was included in the calculation. The numbers separated by colons (on the first page) refer to the number of forms of the protein for that entry: number of tyrosine residues: number of tryptophan residues: and number of phenylalanine residues. The next number is the accession number for the protein in the Swiss-Prot database followed by the name and additional identifiers as included in the database. The second line displays the calculated molecular weight, the third line displays the Y/W ratio (a −1 is reported for entries where there is a 0 in the denominator, and the fourth line displays the Y/F ratio. The penultimate line displays the calculated pI. The end of the report displays a tally of the number of entries found in the search.

### Example 4

Program for Protein Identification

The program for protein identification begins by calculating the molecular weight of each protein in the databank. This is done simply by counting the numbers of each amino acid present, multiplying the number of each type of amino acid by the corresponding molecular weight of each amino acid, and summing these masses to calculate the molecular weight of the intact polypeptide. Molecular weight calculations were performed without the amino terminal methionine and in those cases where the leader peptide was annotated, molecular weight calculations were performed for the intact polypeptide as well as for the peptide minus the leader sequence. Annotated disulfide bonds were included in calculations.

The Y/W ratio was determined by dividing the number of tyrosine residues present by the number of tryptophan residues present and the Y/F ratio was calculated by dividing the number of tyrosine residues by the number of phenylalanine residues present in the polypeptide.

The theoretical isoelectric point of the polypeptide was calculated using the pKa values for amino acid and the algorithm of Skoog and Wickman, 1986 (Skoog, B. and Wichman A., 1986, Trends in Analytical Chemistry, Vol. 5, p. 82). The net charge of the molecule was determined at pH units ranging from 1-11. Successive iterations of 0.05 pH units were used to approximate the pI.

Molecular weight, Y/W and Y/F ratios, and pI values were calculated for proteins from humans, mice, cows, rats, and E. coli using the Swiss-Prot database. Results for each species were saved in look-up tables linked to a web page. The web interface allowed input of measured values for a given sample and a range of uncertainty values were allowed for each parameter. See FIG. 3.

The efficacy of this approach was investigated by determining the number of proteins in the human database (9990 proteins) that could be distinguished using this method. Using script coded for SAS software (Cary, N.C.) the following sets of uncertainty values were assessed. See Table 2.

TABLE 2

| Parameter set | MW+/− | Y/W+/− | Y/F+/− | pI+/− |
|---|---|---|---|---|
| SET 1 | 0.05% | 10% | 10% | 0.5 |
| SET 2 | 10% | 10% | 10% | 0.5 |
| SET 3 | 0.05% | 10% | 10% | 10 |
| SET 4 | 0.05% | 100000 | 100000 | 0.5 |
| SET 5 | 0.01% | 100000 | 100000 | 0.5 |
| SET 6 | 0.01% | 10% | 10% | 0.5 |
| SET 7 | 0.01% | 10% | 10% | 10 |
| SET 8 | 0.001% | 10% | 10% | 0.5 |

Set 1 assumes that MS measurements were made by MALDI-TOF, amino acid ratios where spectrophotometric, pI was from non-immobilized ampholines.
Set 2 assumes both MW and pI measurements were from 2D-PAGE, not MS.
Set 3 assumes no pI information.
Set 4 eliminates amino acid ratios
Set 5 eliminates amino acid ratios, higher precision on MW
Set 6 higher precision on MW
Set 7 higher precision on MW, no pI
Set 8 very high precision on MW, amino acid ratios where spectrophotometric, pI was from non-immobilized ampholines.

Results

| | p95 (95^{th} percentile | PERCENT |
|---|---|---|
| | of hits found | of entries |
| Set | by single seed) | having only 1 hit |
| 1 | 3 | 83.94716 |
| 2 | 13 | 16.94355 |
| 3 | 6 | 61.1189 |
| 4 | 11 | 13.77102 |
| 5 | 11 | 13.81105 |
| 6 | 3 | 83.94716 |
| 7 | 6 | 61.1189 |
| 8 | 3 | 83.94716 |

Table 3 showing 95^{th} percentile of hits found by single seed and the PERCENT of entries having only 1 hit for the Set parameters defined in Table 2

These calculations reveal that the combination of all three measurements (i.e., MW, pI, the ratios of amino acids) being made with practical accuracy give the best differentiation of proteins in the database.

Y/W and Y/F ratios, and pI values are practically measured with variation being +/−10% for ratios and +/−0.5 pH units for pI. Methods for molecular weight measurement differ in the level of accuracy that can be achieved. However, identical results were observed for molecular weight variations of 0.05-0.001% showing that precision in molecular weight determination higher than 0.05% did not improve the numbers of proteins that were individually distinguishable.

### Example 5

Calculation of Uncertainty for the Method of the Instant Invention

The inventors determined the uncertainty factors for the amino acid ratios and the pI parameters by the range of accuracy that the inventors believed to be practical given the present state of the art for technologies used in their determination.

Calculation of Y/W and Y/F was considered to be subject to 10% uncertainty (i.e. +/−0.1 times the measured ratio) and the pI was considered to be estimable within 1 pH unit (i.e. +/−0.5 pH units).

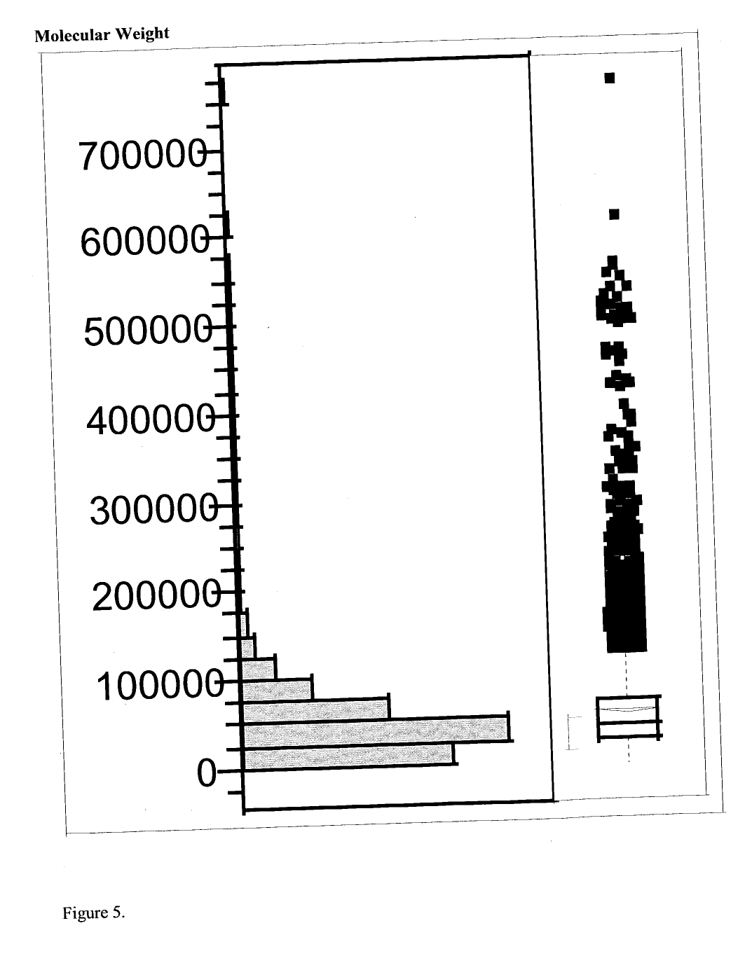

See FIG. 5 for the distribution of Molecular Weights of proteins in the Swiss-Prot database. Table 4 below displays the quantiles for the distribution of Molecular Weights of proteins in the Swiss-Prot database. Table 5 below displays relevant data for the proteins for the Molecular Weights of proteins in the Swiss-Prot database. The distributions of molecular weights of the proteins in the Swiss-Prot database are sufficiently disperse that simply by combining a molecular weight determination (with some flexibility allowed) with one other physical parameter or more physical parameters allows one to uniquely identify or identify a small set of proteins. FIG. 5, Table 4, and Table 5 show that most of the proteins in the Swiss-Prot database have a molecular weight of less than 200,000 daltons (>97.5%) with a mean of roughly 54,000 daltons. Table 4 shows that the molecular weight of the protein with the most mass is over 770,000 daltons.

TABLE 4
Quantiles for the distribution of Molecular Weights
of proteins in the Swiss-Prot database
Quantiles

| | | |
|---|---|---|
| 100.0% | maximum | 773087 |
| 99.5% | | 341540 |
| 97.5% | | 187271 |
| 90.0% | | 104723 |
| 75.0% | quartile | 66530 |
| 50.0% | median | 41350 |
| 25.0% | quartile | 23848 |
| 10.0% | | 12432 |
| 2.5% | | 5034 |
| 0.5% | | 1049 |
| 0.0% | minimum | 204 |

TABLE 5
Relevant data of the proteins in the Swiss-Prot database
regarding their Molecular Weights.
Moments

| | |
|---|---|
| Mean | 54257.297 |
| Std Dev | 52527.739 |
| Std Err Mean | 525.54023 |
| upper 95% Mean | 55287.461 |
| lower 95% Mean | 53227.132 |
| N | 9990 |

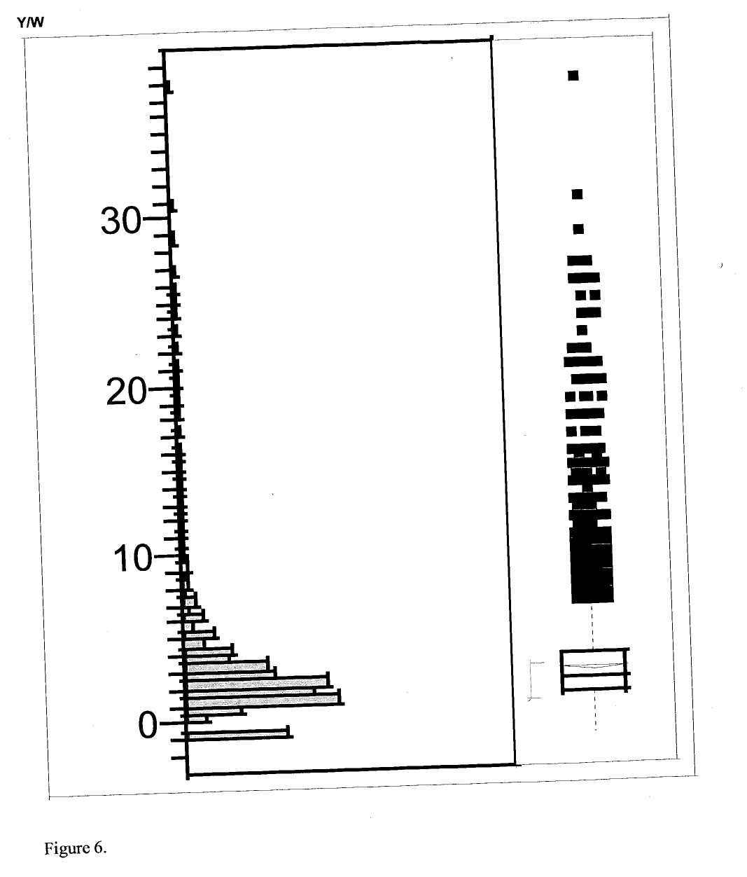

See FIG. 6 for the distribution of tyrosine to tryptophan ratio in proteins in the Swiss-Prot database. Table 6 below displays the quantiles for the distribution of tyrosine to tryptophan ratio of proteins in the Swiss-Prot database. Table 7 below displays statistical data for the tyrosine to tryptophan ratio of the proteins in the Swiss-Prot database.

TABLE 6
Quantiles for the distribution of tyrosine to tryptophan
ratio of proteins in the Swiss-Prot database.
Quantiles

| | | |
|---|---|---|
| 100.0% | maximum | 38.00 |
| 99.5% | | 17.05 |
| 97.5% | | 10.00 |
| 90.0% | | 5.66 |
| 75.0% | quartile | 3.50 |
| 50.0% | median | 2.05 |
| 25.0% | quartile | 1.22 |
| 10.0% | | 0.00 |
| 2.5% | | −1.00 |
| 0.5% | | −1.00 |
| 0.0% | minimum | −1.00 |

TABLE 7
The statistical data for the tyrosine to tryptophan ratio
of the proteins in the Swiss-Prot database.
Moments

| | |
|---|---|
| Mean | 2.6673664 |
| Std Dev | 2.8915051 |
| Std Err Mean | 0.0289295 |
| upper 95% Mean | 2.7240741 |
| lower 95% Mean | 2.6106587 |
| N | 9990 |

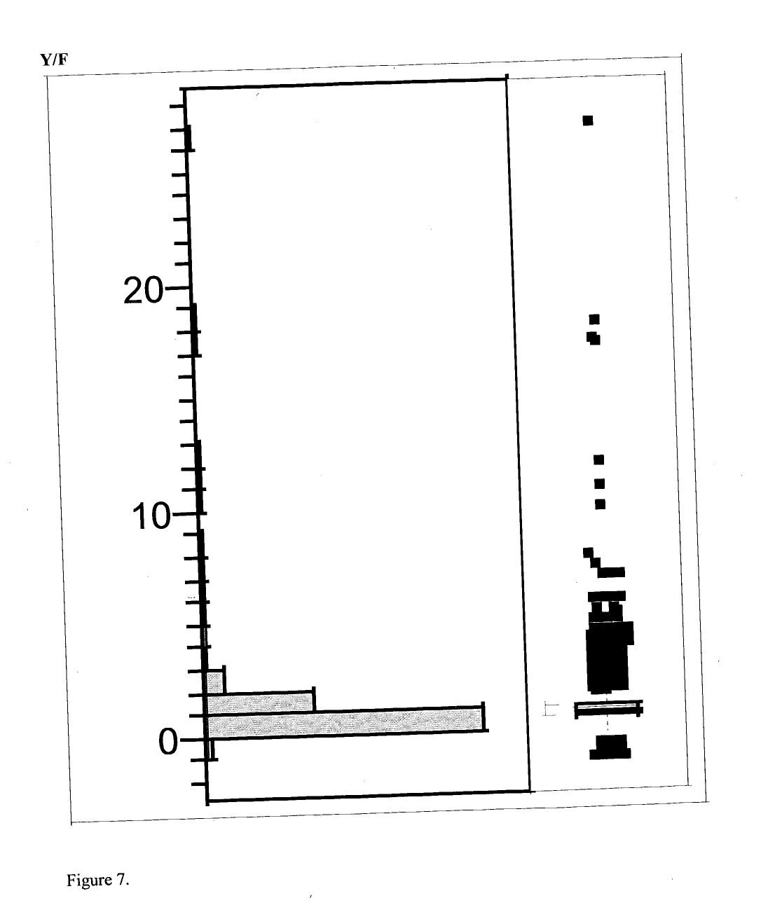

See FIG. 7 for the distribution of tyrosine to phenylalanine ratio in proteins in the Swiss-Prot database. Table 8 below displays the quantiles for the distribution of tyrosine to phenylalanine ratio of proteins in the Swiss-Prot database. Table 9 below displays statistical data for the tyrosine to phenylalanine ratio of the proteins in the Swiss-Prot database.

TABLE 8
Quantiles for the distribution of tyrosine to phenylalanine
ratio of proteins in the Swiss-Prot database.
Quantiles

| | | |
|---|---|---|
| 100.0% | maximum | 27.00 |
| 99.5% | | 4.66 |
| 97.5% | | 2.75 |
| 90.0% | | 1.66 |
| 75.0% | quartile | 1.08 |
| 50.0% | median | 0.76 |
| 25.0% | quartile | 0.53 |
| 10.0% | | 0.33 |
| 2.5% | | 0.00 |
| 0.5% | | −1.00 |
| 0.0% | minimum | −1.00 |

TABLE 9
The statistical data for the tyrosine to phenylalanine ratio of
the proteins in the Swiss-Prot database.
Moments

| | |
|---|---|
| Mean | 0.903977 |
| Std Dev | 0.8408658 |
| Std Err Mean | 0.0084129 |
| upper 95% Mean | 0.9204679 |
| lower 95% Mean | 0.8874861 |
| N | 9990 |

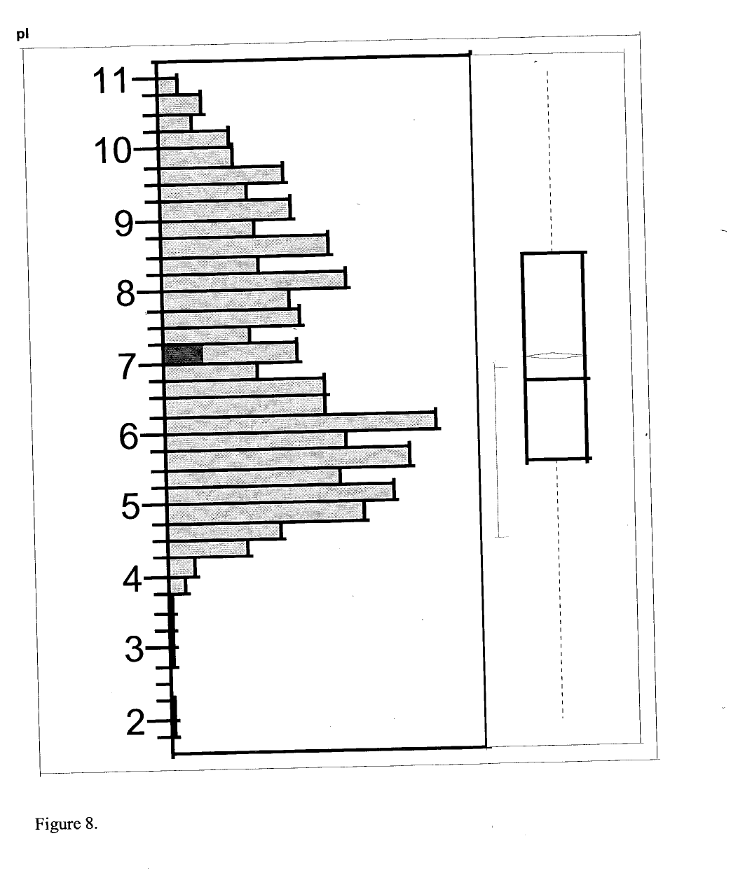

See FIG. 8 for the distribution of isoelectric points in proteins in the Swiss-Prot database. Table 10 below displays the quantiles for the distribution of isoelectric points of proteins in the Swiss-Prot database. Table 11 below displays statistical data for the isoelectric points of the proteins in the Swiss-Prot database. The relatively uniform distribution of isoelectric points for proteins in the Swiss-Prot database makes it an ideal parameter for uniquely identifying a protein from a database.

TABLE 10
Quantiles for the distribution of isoelectric points of
proteins in the Swiss-Prot database.
Quantiles

| | | |
|---|---|---|
| 100.0% | maximum | 10.940 |
| 99.5% | | 10.840 |
| 97.5% | | 10.340 |
| 90.0% | | 9.540 |
| 75.0% | quartile | 8.440 |
| 50.0% | median | 6.640 |
| 25.0% | quartile | 5.540 |
| 10.0% | | 4.840 |
| 2.5% | | 4.350 |
| 0.5% | | 3.850 |
| 0.0% | minimum | 1.950 |

TABLE 11
Statistical data for the isoelectric points of the
proteins in the Swiss-Prot database.
Moments

| | |
|---|---|
| Mean | 6.9824434 |
| Std Dev | 1.7501987 |
| Std Err Mean | 0.0175107 |
| upper 95% Mean | 7.016768 |
| lower 95% Mean | 6.9481189 |
| N | 9990 |

We claim:

1. A method for identifying a purified protein comprising the steps of:
a) determining an amount per molecule of at least one amino acid type but fewer than 18 amino acid types,
b) determining a mass of the protein,
c) submitting a query to a protein database including the mass of the protein and the amount per molecule of at least one amino acid type and
d) identifying the protein based on the mass of the protein and the amount per molecule of at least one amino acid type;
wherein the steps a) and b) are performed in any order.

2. The method of claim 1, wherein said at least one amino acid type comprises at least one aromatic amino acid or cysteine.

3. The method of claim 2, wherein the aromatic amino acid is selected from the group consisting of tyrosine, tryptophan, and phenylalanine.

4. The method of claim 1, 2 or 3 wherein the amount of at least a first amino acid type and a second amino acid type are determined.

5. The method of claim 4 wherein at least one ratio is calculated between the amount per molecule of at least one amino acid type and the amount per molecule of at least a second amino acid type and said ratio is input into the protein database to identify the protein.

6. The method of claim 1 wherein the mass of the protein is determined by a method selected from the group consisting of a mass spectroscopic method, a gel electrophoretic method, and a gel filtration method under denaturing conditions.

7. The method of claim 6 wherein the mass spectroscopic method is selected from the group consisting of MALDI-TOF and electrospray mass spectroscopy.

8. The method of claim 1 wherein the protein database is Swiss-Prot.

9. The method of claim 2 wherein at least one amino acid is cysteine and the amount of cysteine is determined by a spectrofluorometric method.

10. The method of claim 2 wherein the amount of cysteine is determined by a spectrofluorometric method.

11. The method of claim 1 further comprising the step of determining an isoelectric point of the protein and inputting said isoelectric point into the protein database.

12. The method of claim 1, wherein full-length protein is analyzed.

13. The method of claim 1, wherein the amount per molecule of at least one amino acid type is determined to an accuracy that differs by 10% or less from the actual amount per molecule of at least one amino acid type.

14. A method for identifying a purified protein comprising the steps of:
a) determining an amount per molecule of at least one amino acid type but fewer than 18 amino acid types,
b) determining an isoelectric point of the protein,
c) submitting a query to a protein database including the isoelectric point of the protein and the amount per molecule of at least one amino acid type and
d) identifying the protein based on the isoelectric point of the protein and the amount per molecule of at least one amino acid type;
wherein the steps a and b are performed in any order.

15. The method of claim 14 wherein said at least one amino acid type comprises at least one aromatic amino acid or cysteine.

16. The method of claim 15, wherein the aromatic amino acid is selected from the group consisting of tyrosine, tryptophan, and phenylalanine.

17. The method of claim 14, 15 or 16 wherein the amount of at least a first amino acid type and a second amino acid type are determined.

18. The method of claim 17 wherein at least one ratio is calculated between the amount per molecule of a first amino acid type and the amount per molecule of a second amino acid type and said ratio is input into the protein database to identify the protein.

19. The method of claim 14 wherein the protein database is Swiss-Prot.

20. The method of claim 14 wherein at least one amino acid is cysteine and the amount of cysteine is determined by a spectrofluorometric method.

21. The method of claim 15 wherein the amount of cysteine is determined by a spectrofluorometric method.

22. The method of claim 14, wherein full-length protein is analyzed.

23. The method of claim 14, wherein the amount per molecule of at least one amino acid type is determined to an accuracy that differs by 10% or less from the actual amount per molecule of at least one amino acid type.

24. A method for identifying a protein comprising the steps of:
a) submitting a query to a protein database including a mass of the protein and an amount per molecule of at least one amino acid type and
b) identifying the protein based on the mass of the protein and the amount per molecule of at least one amino acid type;
wherein the at least one amino acid type is selected from the group consisting of tyrosine, tryptophan, phenylalanine, and cysteine.

25. The method of claim 24, wherein the at least one amino acid type is selected from the group consisting of tyrosine, tryptophan, and phenylalanine.

26. The method of claim 24, wherein the amount per molecule of at least one amino acid type is determined to an accuracy that differs by 10% or less from the actual amount per molecule of at least one amino acid type.

27. A method for identifying a protein comprising the steps of:
a) submitting a query to a protein database including an isoelectric point of a protein and an amount per molecule of at least one amino acid type and
b) identifying the protein based on the mass of the protein and the amount per molecule of at least one amino acid type;
wherein the at least one amino acid type is selected from the group consisting of tyrosine, tryptophan, phenylalanine, and cysteine.

28. The method of claim 27, wherein the at least one amino acid type is selected from the group consisting of tyrosine, tryptophan, and phenylalanine.

29. The method of claim 27, wherein the amount per molecule of at least one amino acid type is determined to an accuracy that differs by 10% or less from the actual amount per molecule of at least one amino acid type.

30. A method for identifying a protein comprising the steps of:
a) submitting a query to a protein database including a mass of the protein, an isoelectric point of a protein and an amount per molecule of at least one amino acid type and
b) identifying the protein based on the mass of the protein, the isoelectric point of the protein, and the amount per molecule of at least one amino acid type;
wherein the at least one amino acid type is selected from the group consisting of tyrosine, tryptophan, phenylalanine, and cysteine.

31. The method of claim 30, wherein the at least one amino acid type is selected from the group consisting of tyrosine, tryptophan, and phenylalanine.

32. The method of claim 30, wherein the amount per molecule of at least one amino acid type is determined to an accuracy that differs by 10% or less from the actual amount per molecule of at least one amino acid type.
